(19) United States
(12) Patent Application Publication
(10) Pub. No.: US 2026/0297482 A1
(43) Pub. Date: Oct. 1, 2026

(54) MICROBE-GUT-JOINT AXIS MULTI-ORGAN CHIP AND PREPARATION METHOD THEREOF

(71) Applicant: Center for Neuromusculoskeletal Restorative Medicine Limited, Hong Kong (CN)

(72) Inventors: Zhong LI, Hong Kong (CN); Jun LIU, Zhengzhou (CN); Rocky Sung-chi TUAN, Hong Kong (CN)

(73) Assignee: Center for Neuromusculoskeletal Restorative Medicine Limited, Hong Kong (CN)

(21) Appl. No.: 19/629,687

(22) Filed: Mar. 26, 2026

(30) Foreign Application Priority Data
Mar. 27, 2025 (CN) 202510374328.8

(51) Int. Cl. C12M 3/00; B33Y 80/00; C12M 1/00; C12M 3/06; C12N 1/20; C12N 5/071; C12N 5/0775; C12N 5/09

(52) U.S. Cl. CPC C12M 21/08; B33Y 80/00; C12M 23/16; C12M 29/00; C12M 29/04; C12M 29/10; C12M 29/12; C12N 1/20; C12N 5/0663; C12N 5/069; C12N 5/0693

(57) ABSTRACT

The present invention discloses a microbe-gut-joint axis multi-organ chip, comprising: an upper-layer chip, comprising a microbe-gut symbiosis module; a lower-layer chip, comprising a cartilage organoid module; a middle-layer chip, located between the upper-layer chip and the lower-layer chip, comprising a vascular microchannel module and a synovial organoid module; a porous membrane, disposed between the upper-layer chip and the middle-layer chip; and at least one microvalve, configured to dynamically control liquid flow between specific modules within the chip; wherein the microbe-gut symbiosis module and the vascular microchannel module overlap partially in space, and are physically separated by the porous membrane. The invention also relates to a method of preparing the disclosed microbe-gut-joint axis multi-organ chip.

The present application claims priority from CN Patent Application No. 202510374328.8 filed on Mar. 27, 2025, “MICROBE-GUT-JOINT AXIS MULTI-ORGAN CHIP AND PREPARATION METHOD THEREOF,” the content of which is hereby incorporated by reference in its entirety.

### TECHNICAL FIELD

The present invention relates to the field of biomedical engineering, and in particular to a microbe-gut-joint axis multi-organ chip and preparation method thereof.

### BACKGROUND OF THE INVENTION

Osteoarthritis (OA) is one of the most common types of arthritis, and is a chronic degenerative and disabling disease that leads to major health, economic, and social problems, but research work to date has not been able to determine its exact cause. Corresponding treatment strategies for OA are relatively limited, and most are symptomatic treatments, including moderate exercise and rehabilitation strategies, drug interventions, and joint replacement surgeries for late-stage OA. Currently, there is no treatment method that can satisfactorily stop or delay progression of osteoarthritis or provide effective and long-lasting symptom relief.

Gut microbiota (GM) as a key environmental factor widely participates in multiple physiological processes of growth and development and inflammatory lesions of bones and joints. A relationship between gut microbes and joints is called a “microbe-gut-joint axis,” which encompasses complex pathways and mechanisms of interaction between gut microbes and joints. Studying the microbe-gut-joint axis helps to discover potential targets for treating arthritis, and is expected to improve joint system diseases by regulating gut microbiota.

Currently, mechanisms of influence of gut microbes on arthritis remain unclear, mainly due to a lack of effective in vitro models. Widely applied animal models have obvious limitations and defects. In addition to experimental ethics and naturally existing genetic barriers, compositions of gut microbiota of animals and humans also have huge differences. Gut microbes of simple model animals are mostly aerobic, whereas in human intestines, obligate anaerobic bacteria occupy a dominant position. Animal models cannot recapitulate characteristics of human gut microbes in terms of quantity, type, and complexity; secondly, animals and human joints have huge differences in structure, composition, function, reaction, stress, and other aspects, making it very difficult for experimental results of animal models to be translated to human patients. Furthermore, in clinical studies, huge differences also exist in age, gender, physiological conditions, disease degrees, gut microbial compositions, diets, and supplements of subjects. And a heterogeneity of clinical samples is large, and a research time span is generally long.

However, joint research on human patients can only be conducted relying on limited means, with a limited number of biopsy samples and difficulties in conducting follow-up research. Therefore, constructing an effective in vitro microbe-gut-joint axis model is key to studying influences of gut microbiota on joint development, degradation, inflammation, and other aspects.

### SUMMARY OF THE INVENTION

The objective of the present invention is to solve the defects existing in the prior art, and provide a microbe-gut-joint axis multi-organ chip and preparation method thereof.

In accordance with a first aspect of the present invention, there is provided a microbe-gut-joint axis multi-organ chip, wherein the multi-organ chip comprises: an upper-layer chip, comprising a microbe-gut symbiosis module; a lower-layer chip, comprising a cartilage organoid module; a middle-layer chip, located between the upper-layer chip and the lower-layer chip, comprising a vascular microchannel module and a synovial organoid module; a porous membrane, disposed between the upper-layer chip and the middle-layer chip; and at least two microvalve, configured to dynamically control liquid flow between specific modules within the chip; wherein the microbe-gut symbiosis module and the vascular microchannel module overlap partially in space, and are physically separated by the porous membrane. The on-off states of the two microvalves may be different, to facilitate connecting or separating different modules.

In accordance with the first aspect, the upper-layer chip has a thickness of 2 mm, the middle-layer chip has a thickness of 200 μm, and the lower-layer chip has a thickness of 8 mm; the porous membrane has a width of 6 mm, a length of 10 mm, a thickness of 30 μm, a hole diameter of 8 μm, and a center distance between circular holes of 30 μm.

In accordance with the first aspect, the synovial organoid module and the cartilage organoid module overlap structurally, and circular structures of both are concentric circles, wherein a diameter of the circular structure of the synovial organoid module is greater than a diameter of the circular structure of the cartilage organoid module.

In accordance with the first aspect, the microbe-gut symbiosis module comprises a curved upper-layer microchannel and two curved upper-layer vacuum chambers respectively located on both sides of the upper-layer microchannel; both ends of the upper-layer microchannel are respectively connected to an inlet and an outlet of the upper-layer microchannel, and the two upper-layer vacuum chambers are respectively connected to connection ports; the upper-layer microchannel has a width of 750 μm, and a thickness of 500 μm; each of the upper-layer vacuum chambers has a width of 750 μm, and a thickness of 500 μm.

In accordance with the first aspect, the vascular microchannel module comprises a curved middle-layer microchannel connected to an inlet and an outlet, the middle-layer microchannel has a width of 750 μm and a thickness of 200 μm; the synovial organoid module comprises four array structures, and between an inner circle and an outer circle of a single structural unit, there are two sets of trapezoidal arrays respectively comprising two rows of staggered trapezoids.

In accordance with the first aspect, the two sets of trapezoidal arrays are configured to form a physical barrier utilizing surface tension to confine an un-crosslinked hydrogel and cell mixture within the synovial organoid module, thereby preventing the mixture from leaking into the vascular microchannel module or the cartilage organoid module prior to curing.

In accordance with the first aspect, the cartilage organoid module comprises eighteen conical culture units with spherical bottoms and a channel connecting the culture units, each of the conical culture units has a conical depth of 6 mm, a maximum diameter of 48 mm, and the spherical bottom has a spherical diameter of 3 mm.

In accordance with the first aspect, the at least one microvalve has a square channel cross-section, with both width and height being 0.2 mm, and the microvalve channel has a length of 2 mm; the at least one microvalve is of a split design, comprising a rotatable switch, and a channel whose closing and opening are regulated by the switch, and the switch can control a blade in the channel to rotate after being inserted into a center of the channel.

In accordance with the first aspect, a microchannel in the upper-layer chip has a height (e.g. 500 μm) greater than that of a microchannel in the middle-layer chip (e.g. 200 μm), and such a height difference is configured to support formation of an anoxic-oxic interface on both sides of the porous membrane during fluid perfusion.

In accordance with the first aspect, the vacuum chambers described above are configured to connect to a pulsatile vacuum pump to apply cyclic mechanical stretch to the upper-layer microchannel, thereby simulating physiological intestinal peristalsis.

In accordance with the first aspect, the at least one microvalve is disposed between the vascular microchannel module and the synovial organoid module, and is configured to dynamically open at a specific time point to allow microbial metabolites to enter the synovial organoid module from the vascular microchannel module.

In accordance with the first aspect, the upper-layer chip, the middle-layer chip, the lower-layer chip and the porous membrane are made of polydimethylsiloxane (PDMS). The PDMS has a hydrophobicity sufficient to prevent cells from attaching to walls of the cartilage organoid module, thereby promoting gravity-driven self-aggregation of cells at the bottom of the cartilage organoid module.

In accordance with the first aspect, the chip further comprises a biological co-culture system loaded therein. Preferably, the biological co-culture system comprises:
an intestinal epithelial cell layer forming a villus-crypt structure and a mucus layer on an upper surface of the porous membrane;
a human gut microbiota colonized on the mucus layer within the upper-layer chip;
a vascular endothelial cell layer adhered to a lower surface of the porous membrane within the middle-layer chip;
synovial organoids embedded in a cured hydrogel within the synovial organoid module; and
3D cartilage organoids formed by self-aggregated bone marrow mesenchymal stem cells (BMSCs) within the cartilage organoid module.

In accordance with a second aspect of the present invention, there is provided a method for preparing the microbe-gut-joint axis multi-organ chip in accordance with the first aspect. The method comprises following steps:
I. preparing an upper-layer chip positive mold, a middle-layer chip positive mold, and a lower-layer chip positive mold of a multi-organ chip respectively by utilizing high-precision 3D printing technology;
II. preparing a porous membrane by photolithography processing of a silicon wafer having a cylindrical microarray;
III. preparing an upper-layer chip, a middle-layer chip, and a lower-layer chip; and
IV. preparing two microvalves by utilizing high-precision 3D printing technology;
V. assembling the prepared chips.

In accordance with the second aspect, the processing method of the porous membrane is different from that of the chip's layers.

In accordance with the second aspect, the upper-layer chip positive mold, the middle-layer chip positive mold, and the lower-layer chip positive mold and microvalves in step I are produced by using a computer-aided-design (CAD) tool. In particular, the CAD tool may be SolidWorks which generates 3D files in AutoCAD file standards. The positive mold of porous membrane is processed by photolithography. Preferably, CAD may be used for 2D design, and then 3D printing files may be generating using SolidWorks, and after that, 3D printing may be proceeded.

In accordance with the second aspect, step III comprises the following steps:
S1.1: pouring PDMS prepolymer into the upper-layer chip positive mold, the middle-layer chip positive mold, the lower-layer chip positive mold and the positive mold of porous membrane respectively;
S1.2: after pouring, degassing under −80 kPa until all bubbles are removed; and
S1.3: after removing the bubbles, respectively curing the upper-layer chip and the middle-layer chip under a condition of 60° C. for more than 6 h, and curing the lower-layer chip under a condition of 60° C. for more than 10 h.

In accordance with the second aspect, step IV comprises the following steps:
S2.1: after the upper-layer chip is demolded and punched, performing plasma treatment together with the porous membrane, resulting in irreversible bonding;
S2.2: aligning the upper-layer chip bonded with the porous membrane with the middle-layer chip that has not been demolded and performing plasma bonding, and demolding when the plasma bonding is completed; and
S2.3: after punching a hole in a bonded body of the upper-layer and middle-layer chips, aligning it with the lower-layer chip and performing plasma bonding.

In accordance with the second aspect, the hole-punching positions of the upper-layer chip in S2.1 comprise: an inlet and an outlet of a microbe-gut symbiosis module, ventilation ports of vacuum chambers on both sides, an inlet and an outlet of a vascular microchannel module, an inlet and an outlet of a synovial organoid module, and an inlet and an outlet of a cartilage organoid module.

In accordance with the second aspect, before the assembly in step IV, the method further comprises: preparing the at least one microvalve of the chip respectively by utilizing high-precision 3D printing technology; and during assembly, inserting a switch of the microvalve from a hole corresponding to the microvalve on the upper-layer chip into a channel portion of the microvalve.

In accordance with the second aspect, the method further comprises a step of establishing an in vitro model utilizing the prepared chip: inoculating gut-related cells in the upper-layer chip, and inoculating vascular and joint-related cells in the middle-layer and lower-layer chips; perfusing deoxygenated culture medium in the upper-layer chip, and simultaneously perfusing oxygenated culture medium in the middle-layer or lower-layer chip, to establish an anoxic-oxic interface on both sides of the porous membrane.

In accordance with the second aspect, the method further comprises: introducing a human gut microbiota into an anoxic environment of the upper-layer chip for co-culture, wherein the gut microbiota comprises obligate anaerobes Bifidobacterium bifidum and Lactobacillus plantae.

In accordance with the second aspect, inoculating the joint-related cells comprises: utilizing gravity to make a bone marrow mesenchymal stem cell suspension spontaneously aggregate at spherical bottoms of the lower-layer chip to form 3D cartilage organoids; and injecting a mixture of synovial cells and hydrogel into the synovial organoid module and curing the mixture by ultraviolet irradiation.

In accordance with the second aspect, at a specific time point during establishment or culturing of the in vitro model, fluid communication between the vascular microchannel module and the synovial organoid module is dynamically controlled by operating the microvalve, to study influences of gut microbial products on a joint model.

In accordance with the second aspect, the step of establishing the anoxic-oxic interface comprises perfusing the deoxygenated culture medium and the oxygenated culture medium. The perfusion may be conducted at a flow rate of 45 μL/h to 50 μL/h. The flow rate maintains a stable oxygen concentration gradient across the porous membrane while washing away unbound microorganisms from the upper-layer chip.

In accordance with the second aspect, inoculating the gut-related cells comprises inoculating Caco-2 cells and perfusing an intestinal epithelial cell culture medium for at least 6 days to induce the Caco-2 cells to establish an intestinal villus-crypt structure and form a mucus layer on the porous membrane prior to introducing the human gut microbiota.

In accordance with the second aspect, the deoxygenated culture medium perfused in the upper-layer chip is free of antibiotics and free of anti-fungal drugs to permit survival and colonization of the human gut microbiota.

In accordance with the second aspect, injecting the mixture of synovial cells and hydrogel comprises resuspending the synovial cells in a GelMA hydrogel solution and injecting the mixture into the synovial organoid module after the bone marrow mesenchymal stem cell suspension has been statically cultured in the cartilage organoid module for 24 hours to form the 3D cartilage organoids.

In accordance with the second aspect, the specific time point for dynamically controlling fluid communication is 3 days after in situ formation of the 3D cartilage organoids, and wherein opening the microvalves allows metabolic products from the human gut microbiota to selectively pass through an intestinal epithelial barrier and a vascular endothelial barrier on the porous membrane to exert pathophysiological influences on the 3D cartilage organoids.

In accordance with a third aspect of the present invention, there is provided a microbe-gut-joint axis multi-organ chip, used for implementing the preparation method of the microbe-gut-joint axis multi-organ chip according to the second aspect, the multi-organ chip comprises an upper-layer chip, a porous membrane, a middle-layer chip, a lower-layer chip, and microvalves;
the upper-layer chip comprises a microbe-gut symbiosis module I, and a thickness of the upper-layer chip is 2 mm;
a width of the porous membrane is 6 mm, a length is 10 mm, a thickness is 30 μm, a hole diameter is 8 μm, and a center distance between circular holes is 30 μm;
the middle-layer chip comprises a vascular microchannel module II and a synovial organoid module III, and a thickness of the middle-layer chip is 200 μm, and a structural thickness of the synovial organoid module III is 200 μm;
the lower-layer chip comprises a cartilage organoid module IV, and a thickness of the lower-layer chip is 8 mm.

In accordance with the third aspect, curved channel portions of the microbe-gut symbiosis module I and the vascular microchannel module II overlap structurally, and are separated by the porous membrane;
the synovial organoid module III and the cartilage organoid module IV overlap structurally, and circular structures of both are concentric circles, wherein a diameter R1 of the inner circular structure of the synovial organoid module III is greater than a diameter R2 of the circular structure of the cartilage organoid module IV.

In accordance with the third aspect, the microbe-gut symbiosis module I comprises an upper-layer microchannel a shaped as a curve and two upper-layer vacuum chambers b and c shaped as curves respectively located on both sides of the upper-layer microchannel a;
both ends of the upper-layer microchannel a are respectively connected to an inlet 1 and an outlet 2 of the upper-layer microchannel, and the two upper-layer vacuum chambers b and c are respectively connected to connection ports 3 and 4;
a width of the upper-layer microchannel a is 750 μm, and a thickness is 500 μm;
a width of the upper-layer vacuum chambers b and c is 750 μm, and a thickness is 500 μm.

In accordance with the third aspect, the vascular microchannel module II comprises
a middle-layer microchannel d shaped as a curve, respectively connected to an inlet 5 and an outlet 6, wherein a width of the middle-layer microchannel d shaped as the curve is 750 μm, and a thickness is 200 μm;
the synovial organoid module III comprises a total of 4 array structures e-1, e-2, e-3, and e-4, wherein e-1 and e-4 have 4 structural units, and e-2 and e-3 have 5 structural units;
both ends of e-1 comprise an inlet 7 and an outlet 8; both ends of e-2 comprise an inlet 9 and an outlet 10; both ends of e-3 comprise an inlet 11 and an outlet 12, and both ends of e-4 comprise an inlet 13 and an outlet 14;
an inner diameter R1 of a single structural unit is 5 mm, and an outer diameter R3 is 7 mm, and between an inner circle and an outer circle of the single structural unit, there are 2 sets of trapezoidal arrays g-1 and g-2;
the trapezoidal arrays g-1 and g-2 respectively comprise 2 rows of staggered trapezoids, a channel structure h-1 is formed between the trapezoidal arrays g-1 and g-2, a channel structure h-2 is formed between the trapezoidal array g-2 and the outer circle, and widths of the channel structure h-1 and the channel structure h-2 are both 300 μm;
for the trapezoids of the trapezoidal arrays g-1 and g-2, a short side length D1 thereof is 50 μm, a long side length D2 is 100 μm, a height H1 is 100 μm, a spacing D3 between the short sides is 30 μm, a distance D4 between the long sides on a side of the inner circle is 50 μm, and a distance D5 between the long sides on a side of the outer circle is 60 μm;
both ends of the synovial organoid module III are respectively connected to an inlet 15 and an outlet 16.

In accordance with the third aspect, the cartilage organoid module IV comprises 18 conical culture units i with spherical bottoms and a channel j connecting the culture units;
the channel j comprises an inlet 17 and an outlet 18;
a conical depth H2 of the conical culture units i is 6 mm, a maximum diameter R2 is 48 mm, and a spherical diameter of the spherical bottoms is 3 mm.

Compared with the prior art, the microbe-gut-joint axis multi-organ chip of the present invention comprises or consists of an upper-layer chip, a porous membrane structure, a middle-layer chip, and a lower-layer chip, and microvalves, and is used for in vitro modeling and pathophysiological research of the microbe-gut-joint axis. The multi-organ chip as disclosed herein can be used for experiments with human-derived cells or organoids. It is believed that the generated experimental results would have a good correlation with humans, and there is no species difference between animal experiments and humans. Moreover, the multi-organ chip adopts microfluidic chip technology to continuously perfuse fluid in microchannels and chambers to culture cells and tissues, which can significantly accelerate experimental processes and shorten experimental time. In addition, the disclosed multi-organ chip combines microfluidic technology and fluid mechanics, and adopts a multi-organ chip processed by PDMS. The use of the disclosed multi-organ chip, instead of performing animal experiments, can greatly save experimental costs, and avoid ethical controversies. This allows it to be scaled up to standardized processing and production for high-throughput, large-scale experimental research.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings are provided to further illustrate the invention and form part of the specification. They are used together with the embodiments of the invention to explain the invention and do not constitute a limitation thereof.

FIG. 1 is a flow chart showing a method for preparing a microbe-gut-joint axis multi-organ chip in accordance with an embodiment of the present invention.

FIG. 2 is a schematic diagram of the chip structure of the microbe-gut-joint axis multi-organ chip in accordance with an embodiment of the present invention.

FIG. 3 is a schematic diagram of the structure of a microbe-gut symbiosis module I of the upper-layer chip of the microbe-gut-joint axis multi-organ chip in accordance with an embodiment of the present invention.

FIG. 4 is a schematic diagram of the channels, i.e. the gut channel and the vacuum channel, of the microbe-gut symbiosis module I of the upper-layer chip of the microbe-gut-joint axis multi-organ chip in accordance with an embodiment of the present invention.

FIG. 5 is a schematic diagram of inlets and outlets of the microbe-gut symbiosis module I of the upper-layer chip of the microbe-gut-joint axis multi-organ chip in accordance with an embodiment of the present invention.

FIG. 6 is a schematic diagram of the structure of a vascular microchannel module II and a synovial organoid module III of a middle-layer chip of the microbe-gut-joint axis multi-organ chip in accordance with an embodiment of the present invention.

FIG. 7 is a schematic diagram of the channels and inlets and outlets of the vascular microchannel module II of the middle-layer chip of the microbe-gut-joint axis multi-organ chip in accordance with an embodiment of the present invention.

FIG. 8 is a schematic diagram of an array structure of the synovial organoid module III of the middle-layer chip of the microbe-gut-joint axis multi-organ chip in accordance with an embodiment of the present invention.

FIG. 9 is a schematic diagram of inlets and outlets of the synovial organoid module III of the middle-layer chip of the microbe-gut-joint axis multi-organ chip in accordance with an embodiment of the present invention.

FIG. 10 is a schematic diagram of a microvalve 1 (μV1) on the vascular microchannel module II and a microvalve 2 (μV2) on the channel of the synovial organoid module III in the intermediate layer chip of the microbial-gut-joint axis multi-organ chip in accordance with an embodiment of the present invention; the microvalve's switch is exposed on the upper layer of the chip, but the working part of the valve is in the middle layer of the chip; and the flow of liquid in the channel can be controlled or stopped by adjusting the switch of the microvalve.

FIG. 11 is a schematic diagram showing the cross-section of the microvalve in accordance with an embodiment of the present invention, in which the microvalve is opened to allow the flow of the liquid in the channel.

FIG. 12 is a schematic diagram showing the cross-section of the microvalve in accordance with an embodiment of the present invention, in which the microvalve is closed to stop the flow of the liquid in the channel.

FIG. 13 is a schematic diagram of structural units of the synovial organoid module III of the middle-layer chip of the microbe-gut-joint axis multi-organ chip in accordance with an embodiment of the present invention.

FIG. 14 is a schematic diagram of a trapezoidal array of the synovial organoid module III of the middle-layer chip of the microbe-gut-joint axis multi-organ chip in accordance with an embodiment of the present invention.

FIG. 15 is a schematic structural diagram of a cartilage organoid module IV of a lower-layer chip of the microbe-gut-joint axis multi-organ chip in accordance with an embodiment of the present invention.

FIG. 16 is a schematic diagram of the structure of an inlet, an outlet, and a culture chamber of the cartilage organoid module IV of the lower-layer chip of the microbe-gut-joint axis multi-organ chip in accordance with an embodiment of the present invention.

FIG. 17 is a schematic diagram of a single culture unit in the cartilage organoid module IV of the lower-layer chip of the microbe-gut-joint axis multi-organ chip in accordance with an embodiment of the present invention.

FIG. 18 is a schematic diagram of the structure of a porous membrane of the microbe-gut-joint axis multi-organ chip in accordance with an embodiment of the present invention.

FIG. 19 is a schematic diagram of the overall structure of a multi-organ chip in accordance with an embodiment of the present invention.

FIG. 20 is a computer simulation diagram of oxygen concentration distribution forming an anoxic-oxic interface under a set flow rate in the multi-organ chip of the present invention.

FIG. 21 is a scanning electron microscope (SEM) image showing Caco-2 cells forming an intestinal villus-crypt structure and colonizing probiotic microbiota on a porous membrane within the chip of the present invention.

FIG. 22 shows laser confocal fluorescence images showing relative positional distributions of an intestinal epithelial cell layer, a mucus layer, and microorganisms (Bifidobacterium bifidum and Lactobacillus plantae) within the multi-organ chip.

### DETAILED DESCRIPTION OF THE EMBODIMENTS

The technical solutions of the embodiments of the present invention will be clearly and completely described below with reference to the accompanying drawings. It would be appreciated that the described embodiments are only some embodiments of the present invention, and not all of the embodiments.

### Example 1

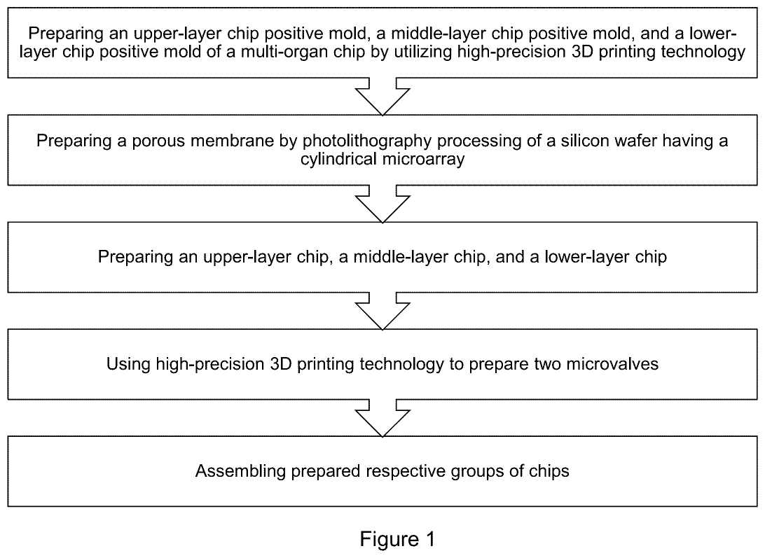

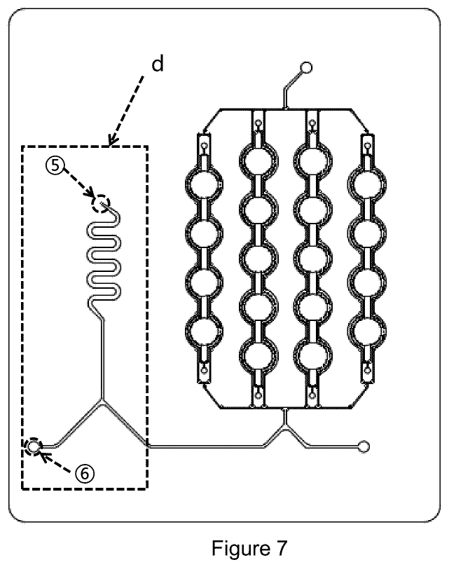

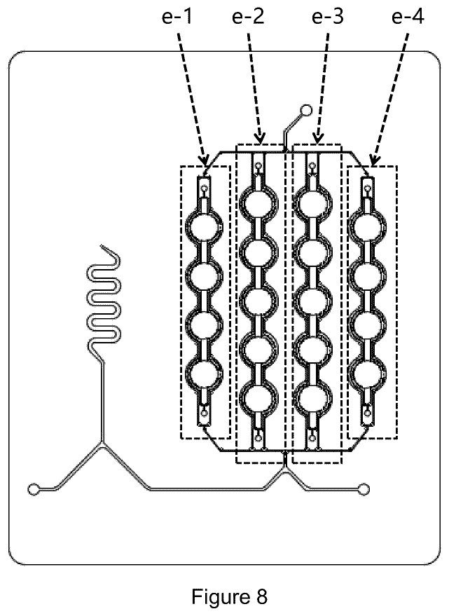

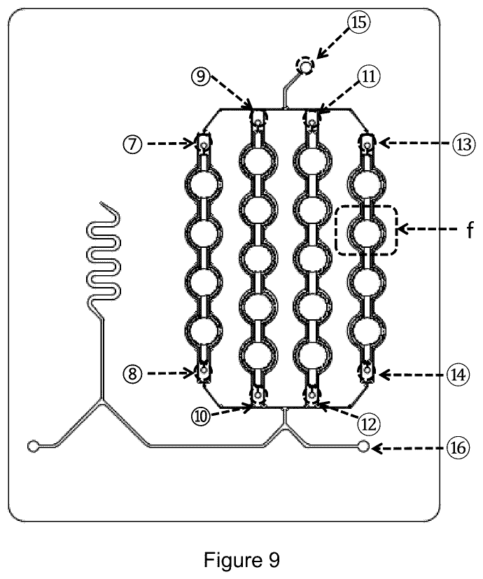

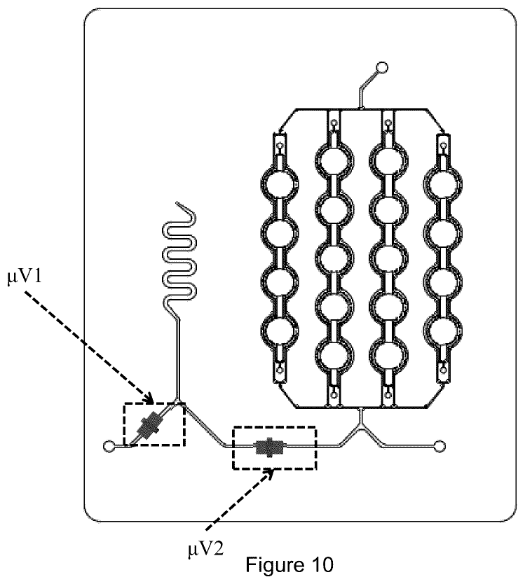

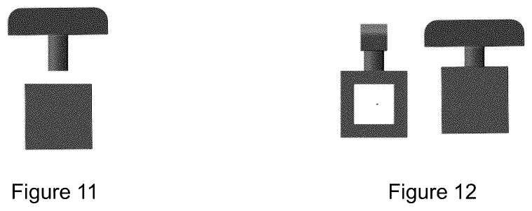

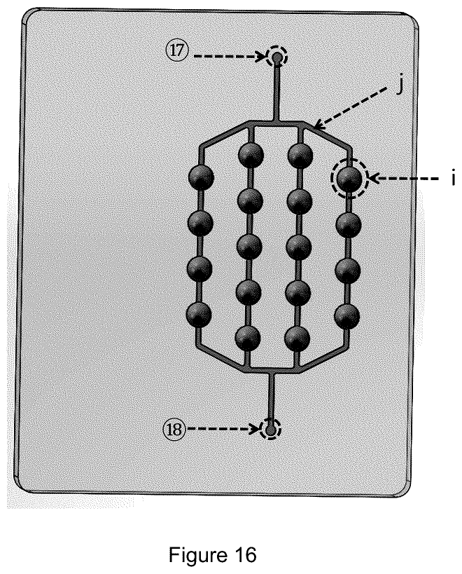

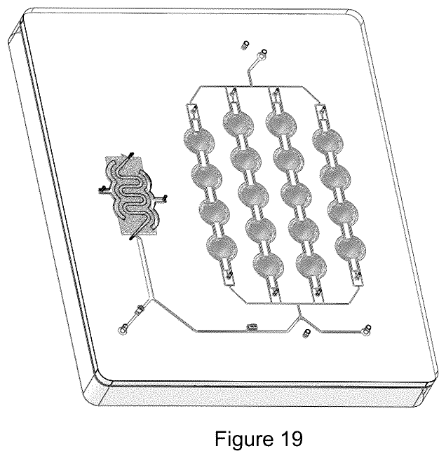

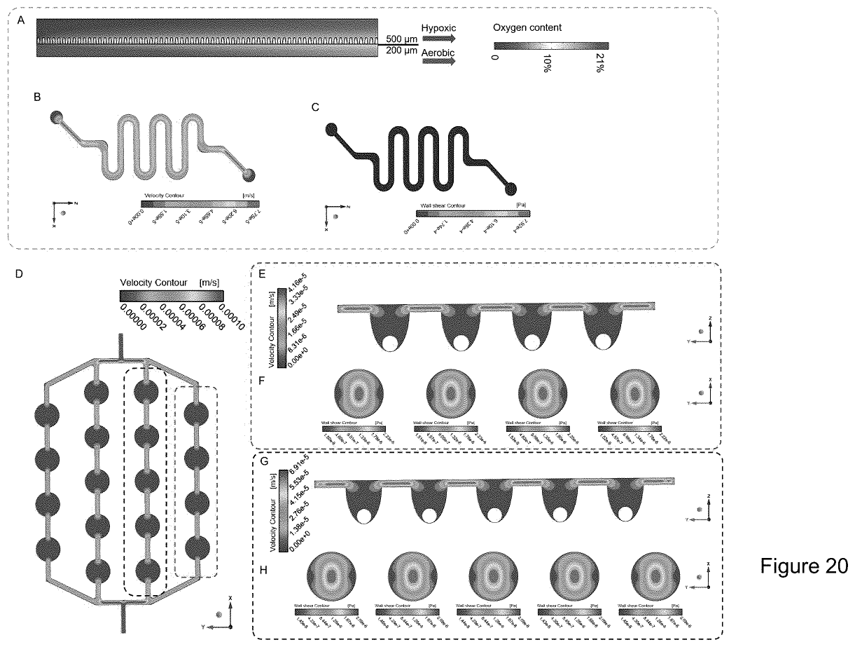

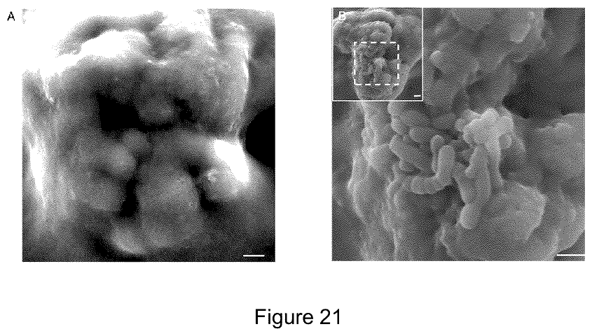

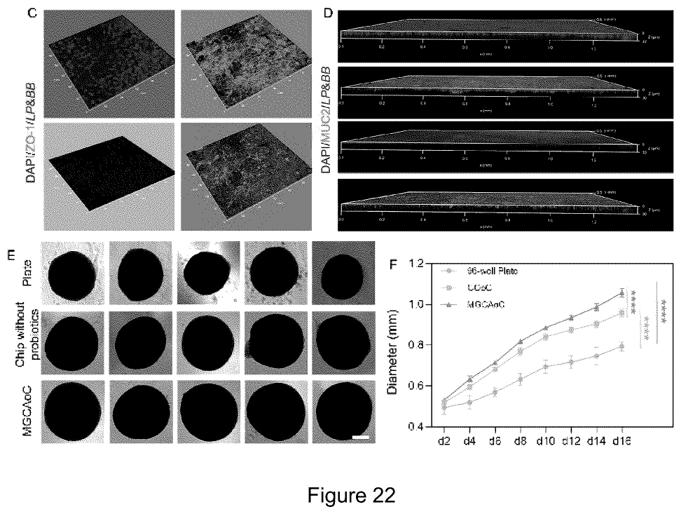

Referring to FIG. 1 to FIG. 22, this example discloses a method for preparing a microbe-gut-joint axis multi-organ chip. In particular, the method comprises the steps of:
I. preparing an upper-layer chip positive mold, a middle-layer chip positive mold, and a lower-layer chip positive mold of a multi-organ chip respectively by utilizing high-precision 3D printing technology;
II. preparing a porous membrane by photolithography processing of a silicon wafer having a cylindrical microarray;
III. preparing an upper-layer chip, a middle-layer chip, and a lower-layer chip; and
IV. preparing two microvalves by utilizing high-precision 3D printing technology;
V. assembling the prepared chips.

It should be noted that the upper-layer chip positive mold, the middle-layer chip positive mold, and the lower-layer chip positive mold take AutoCAD files as standards respectively, and generate 3D files by using Solidworks. In particular, the upper-layer chip has a thickness of 2 mm, the middle-layer chip has a thickness of 0.2 mm, and the lower-layer chip has a thickness of 8 mm. The upper-layer chip comprises a microbe-gut symbiosis module I, the middle-layer chip comprises a vascular microchannel module II and a synovial organoid module III, and the lower-layer chip comprises a cartilage organoid module IV.

Before the assembly, the method further comprises: preparing at least one microvalve of the chip respectively by utilizing high-precision 3D printing technology. In an embodiment, at least one microvalve has a square channel cross-section, with both of the width and height being 0.2 mm. The microvalve channel has a length of 2 mm. Specifically, the at least one microvalve is of a split design, comprising a rotatable switch, and a channel whose closing and opening are regulated by the switch. The switch can control a blade in the channel to rotate after being inserted into a center of the channel.

Specifically, PDMS prepolymer is respectively poured into the upper-layer chip positive mold, the middle-layer chip positive mold, the lower-layer chip positive mold, and the positive mold of porous membrane respectively. After pouring is completed, degassing is performed under −80 kPa until all bubbles are removed. After removing the bubbles, the upper-layer chip and the middle-layer chip are respectively cured under a condition of 60° C. for more than 6 h, and the lower-layer chip is cured under a condition of 60° C. for more than 10 h.

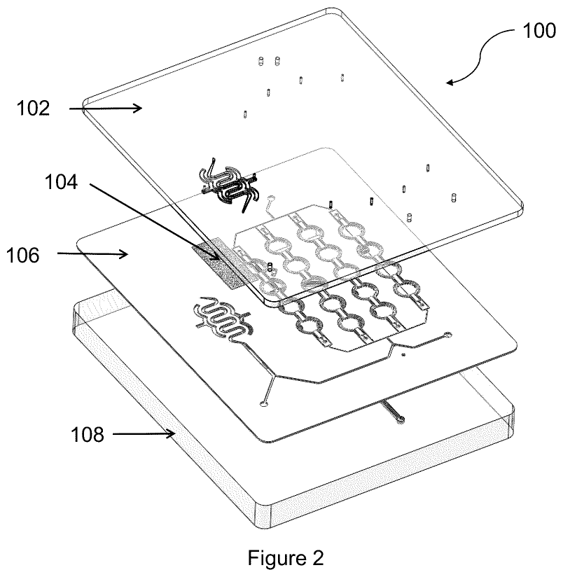

With reference to FIG. 2, the upper-layer chip is then demolded, the hole-punching positions of the upper-layer chip are punched with a punch (e.g. by using a biopsy punch). Afterwards, a plasma treatment is performed on a lower surface of the upper-layer chip, which is a structural opening face, together with the porous membrane. The porous membrane is then irreversibly bonded with a curved channel portion of the microbe-gut symbiosis module I. After performing a plasma treatment on the lower surface of the upper-layer chip bonded with the porous membrane together with the middle-layer chip that has been cured but not demolded, two surfaces are aligned and bonded. During the bonding, the curved channel portions of the microbe-gut symbiosis module I and the vascular microchannel module II overlap. These two modules are separated by the porous membrane.

When the bonding is completed, a middle layer is demolded from the positive mold, and the hole-punching positions on a bonded body of the upper-layer and middle-layer chips are punched with the punch. In particular, the hole-punching positions correspond to inlet and outlet positions on the lower-layer chip. After performing the plasma treatment on lower surfaces of the bonded upper-layer chip and middle-layer chip together with an upper surface structural opening face of the lower-layer chip, two surfaces are aligned and bonded to obtain the microbe-gut-joint axis multi-organ chip, wherein at this time, the synovial organoid module III of the middle layer overlaps with the cartilage organoid module IV of the lower layer, and a circular structure of the synovial organoid module III and a circular structure of the cartilage organoid module IV are concentric circles.

During assembly, a switch of the microvalve is inserted from a hole corresponding to the microvalve on the upper-layer chip into a channel portion of the microvalve.

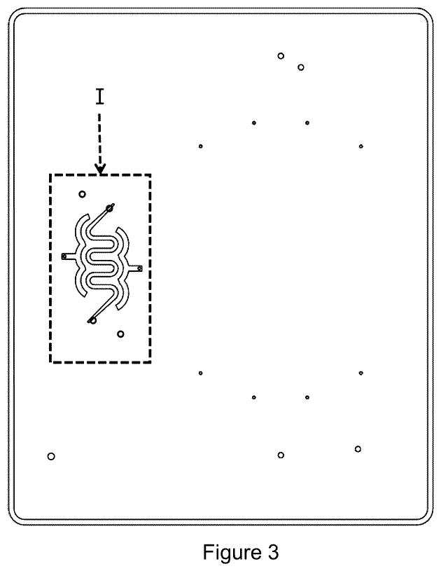

Referring to FIGS. 3-17, the hole-punching positions of the upper-layer chip comprise: an inlet 1 and an outlet 2 of the microbe-gut symbiosis module I corresponding to hole-opening positions of the upper-layer chip; ventilation ports 3 and 4 of vacuum chambers on both sides of the microbe-gut symbiosis module I corresponding to hole-opening positions of the upper-layer chip; an inlet 5 and an outlet 6 of the vascular microchannel module II of the middle-layer chip corresponding to hole-opening positions of the upper-layer chip; an inlet 7, 9, 11, 13, 15 and an outlet 8, 10, 12, 14, 16 of the synovial organoid module III of the middle-layer chip corresponding to hole-opening positions of the upper-layer chip.

The hole-punching positions on the bonded body of the upper-layer and middle-layer chips comprise an inlet 17 and an outlet 18 of the cartilage organoid module IV of the lower-layer chip being located at hole-opening positions of the middle-layer chip.

The method further comprises a step of establishing an in vitro model utilizing the prepared chip: inoculating gut-related cells in the upper-layer chip, and inoculating vascular and joint-related cells in the middle-layer and lower-layer chips.

In particular, after forming the microbe-gut-joint axis multi-organ chip, the chip is inverted, and a mixture of 1% Matrigel and rat tail collagen I, in a ratio of 1:1, is injected from the inlet 5 of the middle-layer chip, so that it fills the entire curved microchannel d of the vascular microchannel module II. After being placed in a 37° C. incubator for 1 h, the mixture of Matrigel and collagen is slowly aspirated out, and human umbilical vein endothelial cells (HUVEC) are digested and resuspended at a density of 1.5×10⁵ cells/cm², then injected into the microchannel d and placed in a 37° C. incubator. After standing for 12 h, the chip is flipped. At this time, HUVEC cells adhere to a lower surface of the porous membrane V. An endothelial cell culture medium is passed into the inlet 5 of the vascular microchannel module II at a flow rate of 50 μL/h.

A mixture of Matrigel and rat tail collagen I, in a ratio of 1:1, is then injected from the inlet 1 of the upper-layer chip, so that it fills the entire curved microchannel a of the microbe-gut module I. After being placed in a 37° C. incubator for 1 h, the mixture of Matrigel and collagen is slowly aspirated out, and human intestinal epithelial cells (Caco-2) are digested and resuspended at a density of 1.5×10⁵ cells/cm², then injected into the microchannel a and left to stand in a 37° C. incubator for 12 h. At this time, Caco-2 cells adhere to an upper surface of the porous membrane V. An intestinal epithelial cell culture medium is passed into the inlet 1 of the microbe-gut module I at a flow rate of 50 μL/h.

Preferably, the fluid perfusion flow rates in the upper-layer chip and the middle-layer chip are controlled at a specific flow rate in a range of 45 μL/h to 50 μL/h. A microchannel in the upper-layer chip has a height (e.g. 500 μm) greater than that of a microchannel in the middle-layer chip (200 μm), and this height difference is configured to support formation of an anoxic-oxic interface on both sides of the porous membrane during fluid perfusion.

Deoxygenated culture medium is perfused in the upper-layer chip, while oxygenated culture medium is perfused simultaneously in the middle-layer or lower-layer chip, thereby maintaining a stable oxygen concentration gradient at the porous membrane separating two layers of channels, establishing an anoxic-oxic interface. By perfusing the deoxygenated culture medium and the oxygenated culture medium at the flow rate of 45 μL/h to 50 μL/h, the flow rate maintains a stable oxygen concentration gradient across the porous membrane while washing away unbound microorganisms from the upper-layer chip. At the same time, the vacuum chambers are configured to connect to a pulsatile vacuum pump. By utilizing the pulsatile vacuum pump connected to ports 3 and 4 of the vacuum chambers of the upper-layer chip, vacuum stimulation is alternately applied at a specific frequency (for example, 1 Hz), thereby applying cyclic mechanical stretch to the upper-layer microchannel a to simulate physiological intestinal peristalsis.

Moreover, the step of inoculating the gut-related cells comprises inoculating Caco-2 cells and perfusing an intestinal epithelial cell culture medium for at least 6 days to induce the Caco-2 cells to establish an intestinal villus-crypt structure and form a mucus layer on the porous membrane prior to introducing the human gut microbiota.

A culture medium free of antibiotics and anti-fungal drugs is perfused in the microchannel a of the microbe-gut module I and the microchannel d of the vascular microchannel module II for 24 h. If co-cultured microorganisms contain anaerobic microorganisms, the culture medium free of antibiotics and anti-fungal drugs perfused for 24 h in the microchannel a of the microbe-gut module I needs to be subjected to deoxygenation treatment. Whereas the culture medium perfused in the microchannel d of the vascular microchannel module II does not need deoxygenation treatment. The deoxygenated culture medium perfused in the upper-layer chip is free of antibiotics and free of anti-fungal drugs to permit survival and colonization of the human gut microbiota.

The method further comprises: introducing a human gut microbiota into an anoxic environment of the upper-layer chip for co-culture. Specifically, a microbial mixture comprising the human gut microbiota, wherein the gut microbiota comprises obligate anaerobes Bifidobacterium bifidum and Lactobacillus plantae, is inoculated in the microchannel a at a density of 1×10⁷ CFU/mL. After culturing, the intestinal epithelial cells Caco-2 establish an intestinal villus-crypt structure on the porous membrane and form a mucus layer, and the above microbiota (anaerobes, etc.) colonizes on a surface of the mucus layer.

Caco-2 culture medium free of antibiotics and anti-fungal drugs (selecting deoxygenated/non-deoxygenated treatment according to whether anaerobic microorganisms are included) continues to be used to perform perfusion culture on the upper-layer microchannel, at a flow rate of 50 μL/h. Then microorganisms not attached to an intestinal epithelial surface will be washed away by the flowing culture medium. At this time, the culture medium in the microchannel a of the microbe-gut module I flows out of the chip through the outlet 2, and the culture medium in the microchannel d of the vascular microchannel module II flows out of the chip through the outlet 6.

The step of inoculating the joint-related cells comprises: utilizing gravity to make a bone marrow mesenchymal stem cell suspension spontaneously aggregate at spherical bottoms of the lower-layer chip to form 3D cartilage organoids; and injecting a mixture of synovial cells and hydrogel into the synovial organoid module, and curing the mixture by ultraviolet irradiation. For example, the digested human bone marrow mesenchymal stem cells (BMSC) are resuspended at a density of 1×10⁶ cells/mL. 1.5 mL of the cell suspension is injected into the cartilage organoid module IV from an inlet 17, and the chip is left to stand in a 37° C. incubator for 24 h. In particular, the upper-layer chip, the middle-layer chip, and the lower-layer chip comprise PDMS. The PDMS has a hydrophobicity sufficient to prevent cells from attaching to walls of the cartilage organoid module, thereby promoting gravity-driven self-aggregation of cells at the bottom of the cartilage organoid module to form spheres.

In the example, after 24 h of incubation, synovial organoids are constructed on the chip. After synovial organoids are successfully constructed, a cartilage culture medium is perfused from the inlet 17 of the channel j connecting the cartilage organoid culture units to induce in situ formation of cartilage organoids, and discarded culture medium flows out from the outlet 18.

A GelMA hydrogel solution with a concentration of 15% is prepared, and a LAP concentration is 0.15%. The step of injecting the mixture of synovial cells and hydrogel comprises resuspending the synovial cells in a GelMA hydrogel solution at a density of 4×10⁶ cells/mL and injecting the mixture into the synovial organoid module after the bone marrow mesenchymal stem cell suspension has been statically cultured in the cartilage organoid module for 24 hours to form the 3D cartilage organoids. Specifically, the mixture of cells and hydrogel is slowly injected from the inlet 7, 9, 11, and 13 of the synovial organoid module III of the middle layer, until the mixture fills culture chambers of 4 array structures e-1, e-2, e-3, and e-4 of the synovial organoid. The trapezoidal array structures on both sides of the culture chambers can effectively restrict the mixture of hydrogel from flowing to channels or cartilage organoid culture chambers. An ultraviolet lamp with a power of 25 W and a wavelength of 365 nm is used to irradiate the chip for 2 min to cure the hydrogel. Afterwards, a synovial cell culture medium is passed into the inlet 15 of the synovial organoid module III at a flow rate of 50 μL/h, and discarded culture medium flows out from the outlet 16.

The microvalves are disposed between the vascular microchannel module and the synovial organoid module. At a specific time point during establishment or culturing of the in vitro model, fluid communication between the vascular microchannel module and the synovial organoid module is dynamically controlled by operating the microvalves, to study influences of gut microbial products on a joint model. The specific time point for dynamically controlling fluid communication is 3 days after in situ formation of the 3D cartilage organoids, and wherein opening the microvalves allows metabolites from the human gut microbiota to selectively pass through an intestinal epithelial barrier and a vascular endothelial barrier on the porous membrane to exert pathophysiological influences on the 3D cartilage organoids. After opening the microvalve, the outlet 6 of the microchannel d of the vascular microchannel module II connects with the inlet 15 of the synovial organoid module III.

### Example 2

Referring to FIGS. 2-18, there is illustrated a microbe-gut-joint axis multi-organ chip 100. The microbe-gut-joint axis multi-organ chip 100 comprises an upper-layer chip 102, a porous membrane 104, a middle-layer chip 106, and a lower-layer chip 108. The upper-layer chip 102 comprises a microbe-gut symbiosis module I, and has a thickness of 2 mm.

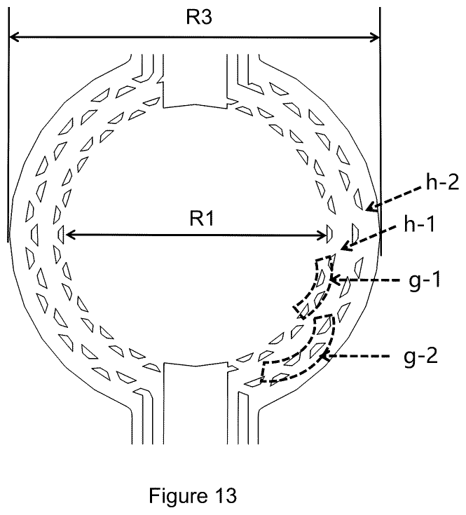

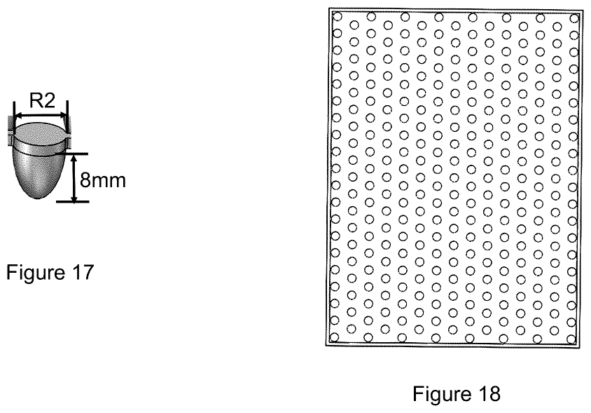

As shown in FIGS. 3-5, the curved channel portions of the microbe-gut symbiosis module I and the vascular microchannel module II overlap structurally, and are separated by the porous membrane 104. The synovial organoid module III and the cartilage organoid module IV overlap structurally, and circular structures of both are concentric circles, wherein a diameter R1 of the inner circular structure of the synovial organoid module III is greater than a diameter R2 of the circular structure of the cartilage organoid module IV, see FIG. 13 and FIG. 17.

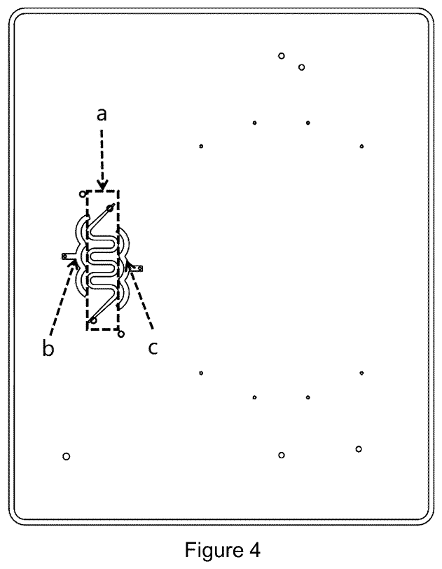

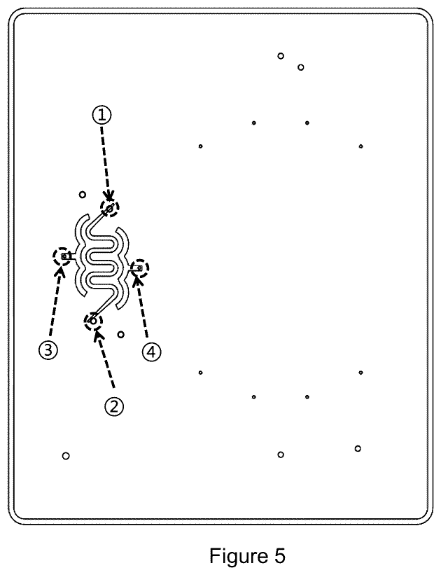

Referring to FIGS. 4-5, the microbe-gut symbiosis module I comprises a curved upper-layer microchannel a and two curved upper-layer vacuum chambers b and c respectively located on both sides of the upper-layer microchannel a, and both ends of the upper-layer microchannel a are respectively connected to an inlet 1 and an outlet 2 of the upper-layer microchannel. The two upper-layer vacuum chambers b and c are respectively connected to connection ports 3 and 4. The upper-layer microchannel a has a width of 750 μm, and a thickness of 500 μm. The upper-layer vacuum chambers b and c have a width of 750 μm, and a thickness of 500 μm.

Referring to FIG. 18, the porous membrane has a width of 6 mm, a length of 10 mm, a thickness of 30 μm, a hole diameter of 8 μm. The center distance between circular holes is 30 μm.

The middle-layer chip 106 comprises a vascular microchannel module II and a synovial organoid module III. In particular, the middle-layer chip has a thickness of 200 μm, and the synovial organoid module III has a structural thickness of 200 μm.

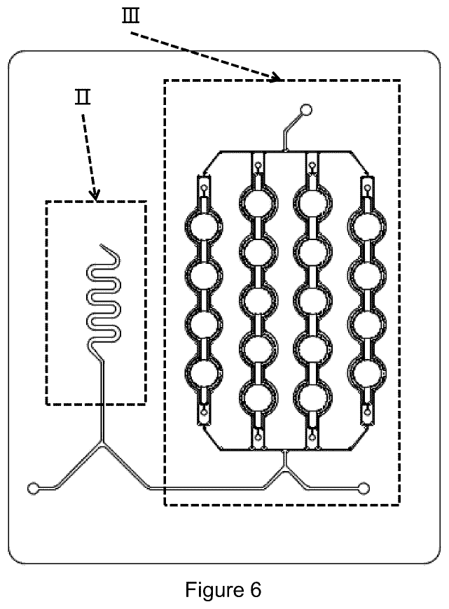

Specifically, referring to FIGS. 6-13, the vascular microchannel module II comprises a curved middle-layer microchannel d, respectively connected to an inlet 5 and an outlet 6. The curved middle-layer microchannel d has a width of 750 μm, and a thickness of 200 μm. The synovial organoid module III comprises a total of four array structures e-1, e-2, e-3, and e-4. In particular, e-1 and e-4 have 4 structural units, and e-2 and e-3 have 5 structural units. The two ends of e-1 comprise an inlet 7 and an outlet 8. The two ends of e-2 comprise an inlet 9 and an outlet 10. The two ends of e-3 comprise an inlet 11 and an outlet 12, and the two ends of e-4 comprise an inlet 13 and an outlet 14.

As shown in FIG. 13, the single structural unit has an inner diameter R1 of 5 mm, and an outer diameter R3 of 7 mm. There are 2 sets of trapezoidal arrays g-1 and g-2 arranged between an inner circle and an outer circle of the single structural unit. Each of the trapezoidal arrays g-1 and g-2 comprises 2 rows of staggered trapezoids. A channel structure h-1 is formed between the trapezoidal arrays g-1 and g-2, and a channel structure h-2 is formed between the trapezoidal array g-2 and the outer circle. Both of the channel structure h-1 and the channel structure h-2 have a width of 300 μm.

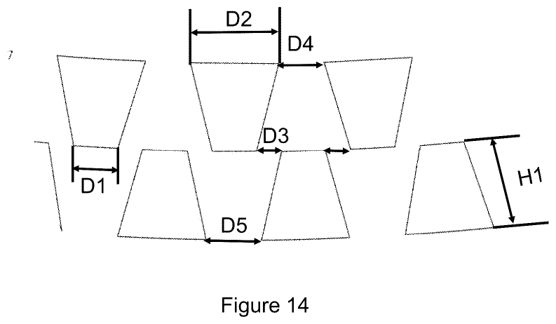

Referring to FIG. 14, for the trapezoids of the trapezoidal arrays g-1 and g-2, a short side length D1 thereof is 50 μm, a long side length D2 is 100 μm, a height H1 is 100 μm, a spacing D3 between the short sides is 30 μm, a distance D4 between the long sides on a side of the inner circle is 50 μm, and a distance D5 between the long sides on a side of the outer circle is 60 μm. The two ends of the synovial organoid module III are respectively connected to an inlet 15 and an outlet 16.

The two sets of trapezoidal arrays g-1 and g-2 are configured to form a physical barrier utilizing surface tension to confine an un-crosslinked hydrogel and cell mixture within the synovial organoid module, thereby preventing the mixture from leaking into the vascular microchannel module or the cartilage organoid module prior to curing.

The lower-layer chip 108 comprises a cartilage organoid module IV, and its thickness is 8 mm.

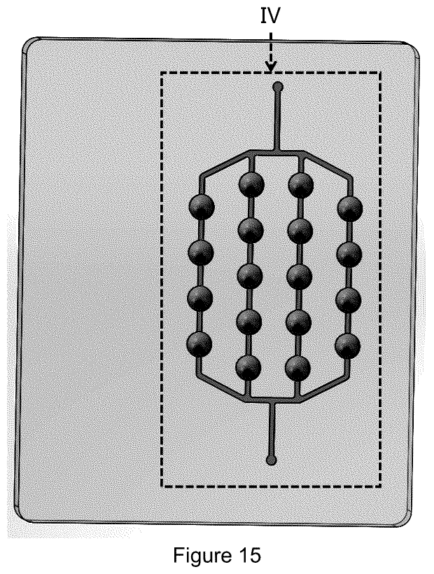

Specifically, referring to FIGS. 15-17, the cartilage organoid module IV comprises eighteen conical culture units i with spherical bottoms and a channel j connecting the culture units. The channel j comprises an inlet 17 and an outlet 18, a conical depth H2 of the conical culture units i is 6 mm, a maximum diameter R2 is 48 mm, and a spherical diameter of the spherical bottoms is 3 mm.

In a working state, the chip further comprises a biological co-culture system loaded therein, the biological co-culture system comprising: an intestinal epithelial cell layer forming a villus-crypt structure and a mucus layer on an upper surface of the porous membrane; a human gut microbiota colonized on the mucus layer within the upper-layer chip; a vascular endothelial cell layer adhered to a lower surface of the porous membrane within the middle-layer chip; synovial organoids embedded in a cured hydrogel within the synovial organoid module; and 3D cartilage organoids formed by self-aggregated mesenchymal stem cells within the cartilage organoid module.

1. A microbe-gut-joint axis multi-organ chip, comprising:
an upper-layer chip, comprising a microbe-gut symbiosis module;
a lower-layer chip, comprising a cartilage organoid module;
a middle-layer chip, located between the upper-layer chip and the lower-layer chip, comprising a vascular microchannel module and a synovial organoid module;
a porous membrane, disposed between the upper-layer chip and the middle-layer chip; and
at least one microvalve, configured to dynamically control liquid flow between specific modules within the chip; wherein the microbe-gut symbiosis module and the vascular microchannel module overlap partially in space, and are physically separated by the porous membrane.

2. The microbe-gut-joint axis multi-organ chip according to claim 1, wherein the upper-layer chip has a thickness of 2 mm, the middle-layer chip has a thickness of 200 μm, and the lower-layer chip has a thickness of 8 mm; the porous membrane has a width of 6 mm, a length of 10 mm, a thickness of 30 μm, a hole diameter of 8 μm, and a center distance between circular holes of 30 μm.

3. The microbe-gut-joint axis multi-organ chip according to claim 1, wherein the synovial organoid module and the cartilage organoid module overlap structurally, and circular structures of both are concentric circles, wherein a diameter of the circular structure of the synovial organoid module is greater than a diameter of the circular structure of the cartilage organoid module.

4. The microbe-gut-joint axis multi-organ chip according to claim 1, wherein the microbe-gut symbiosis module comprises a curved upper-layer microchannel and two curved upper-layer vacuum chambers respectively located on both sides of the upper-layer microchannel; both ends of the upper-layer microchannel are respectively connected to an inlet and an outlet of the upper-layer microchannel, and the two upper-layer vacuum chambers are respectively connected to connection ports; the upper-layer microchannel has a width of 750 μm, and a thickness of 500 μm; each of the upper-layer vacuum chambers has a width of 750 μm, and a thickness of 500 μm.

5. The microbe-gut-joint axis multi-organ chip according to claim 1, wherein the vascular microchannel module comprises a curved middle-layer microchannel connected to an inlet and an outlet, the middle-layer microchannel has a width of 750 μm and a thickness of 200 μm; the synovial organoid module comprises four array structures, and between an inner circle and an outer circle of a single structural unit, there are two sets of trapezoidal arrays respectively comprising two rows of staggered trapezoids.

6. The microbe-gut-joint axis multi-organ chip according to claim 1, wherein the cartilage organoid module comprises eighteen conical culture units with spherical bottoms and a channel connecting the culture units, each of the conical culture units has a conical depth of 6 mm, a maximum diameter of 48 mm, and the spherical bottom has a spherical diameter of 3 mm.

7. The microbe-gut-joint axis multi-organ chip according to claim 1, wherein the at least one microvalve has a square channel cross-section, with both width and height being 0.2 mm, and the microvalve channel has a length of 2 mm; the at least one microvalve is of a split design, comprising a rotatable switch, and a channel whose closing and opening are regulated by the switch, and the switch can control a blade in the channel to rotate after being inserted into a center of the channel.

8. The microbe-gut-joint axis multi-organ chip according to claim 1, wherein a microchannel in the upper-layer chip has a height greater than that of a microchannel in the middle-layer chip, and such a height difference is configured to support formation of an anoxic-oxic interface on both sides of the porous membrane during fluid perfusion.

9. The microbe-gut-joint axis multi-organ chip according to claim 4, wherein the vacuum chambers are configured to connect to a pulsatile vacuum pump to apply cyclic mechanical stretch to the upper-layer microchannel, thereby simulating physiological intestinal peristalsis.

10. The microbe-gut-joint axis multi-organ chip according to claim 1, wherein the at least one microvalve is disposed between the vascular microchannel module and the synovial organoid module, and is configured to dynamically open at a specific time point to allow microbial metabolites to enter the synovial organoid module from the vascular microchannel module.

11. A method for preparing a microbe-gut-joint axis multi-organ chip according to claim 1, comprising:
I. preparing an upper-layer chip positive mold, a middle-layer chip positive mold, and a lower-layer chip positive mold of a multi-organ chip respectively by utilizing 3D printing;
II. preparing a porous membrane by photolithography processing of a silicon wafer having a cylindrical microarray;
III. preparing an upper-layer chip, a middle-layer chip, and a lower-layer chip;
IV. preparing two microvalves by utilizing high-precision 3D printing technology; and
V. assembling the prepared chips.

12. The method according to claim 11, wherein the upper-layer chip positive mold, the middle-layer chip positive mold, the lower-layer chip positive mold, the porous membrane and microvalves in step I are produced by using a computer-aided-design tool.

13. The method according to claim 11, wherein step III comprises:
S1.1: pouring PDMS prepolymer into the upper-layer chip positive mold, the middle-layer chip positive mold, the lower-layer chip positive mold, and the positive mold of porous membrane respectively;
S1.2: after pouring, degassing under −80 kPa until all bubbles are removed;
S1.3: after removing the bubbles, curing the upper-layer chip and the middle-layer chip under a condition of 60° C. for more than 6 h, and curing the lower-layer chip under a condition of 60° C. for more than 10 h.

14. The method according to claim 11, wherein step IV comprises:
S2.1: after the upper-layer chip is demolded and punched, performing plasma treatment together with the porous membrane, resulting in irreversible bonding;
S2.2: aligning the upper-layer chip bonded with the porous membrane with the middle-layer chip that has not been demolded, and performing plasma bonding, and demolding when the plasma bonding is completed;
S2.3: after punching a hole in a bonded body of the upper-layer and middle-layer chips, aligning it with the lower-layer chip and performing plasma bonding.

15. The method according to claim 14, wherein hole-punching positions of the upper-layer chip in S2.1 comprise: an inlet and an outlet of a microbe-gut symbiosis module, ventilation ports of vacuum chambers on both sides, an inlet and an outlet of a vascular microchannel module, an inlet and an outlet of a synovial organoid module, and an inlet and an outlet of a cartilage organoid module.

16. The method according to claim 11, wherein before the assembly in step IV, the method further comprises: preparing the at least one microvalve of the chip respectively by utilizing high-precision 3D printing technology; and during assembly, inserting a switch of the microvalve from a hole corresponding to the microvalve on the upper-layer chip into a channel portion of the microvalve.

17. The method according to claim 11, further comprises a step of establishing an in vitro model utilizing the prepared chip: inoculating gut-related cells in the upper-layer chip, and inoculating vascular and joint-related cells in the middle-layer and lower-layer chips; perfusing deoxygenated culture medium in the upper-layer chip, and simultaneously perfusing oxygenated culture medium in the middle-layer or lower-layer chip, to establish an anoxic-oxic interface on both sides of the porous membrane.

18. The method according to claim 17, further comprises: introducing a human gut microbiota into an anoxic environment of the upper-layer chip for co-culture, wherein the gut microbiota comprises obligate anaerobes Bifidobacterium bifidum and Lactobacillus plantae.

19. The method according to claim 17, wherein inoculating the joint-related cells comprises: utilizing gravity to make a bone marrow mesenchymal stem cell suspension spontaneously aggregate at spherical bottoms of the lower-layer chip to form a 3D cartilage organoid; and injecting a mixture of synovial cells and hydrogel into the synovial organoid module, and curing the mixture by ultraviolet irradiation.

20. The method according to claim 17, wherein at a specific time point during establishment or culturing of the in vitro model, fluid communication between the vascular microchannel module and the synovial organoid module is dynamically controlled by operating the microvalve.
